(19) United States
(12) Patent Application Publication
(10) Pub. No.: US 2004/0249279 A1
(43) Pub. Date: Dec. 9, 2004

(54) Patient monitor for processing signals from an ultrasound probe

(76) Inventor: Michael Maschke, Lonnerstadt (DE)

Correspondence Address: SCHIFF HARDIN LLP, Patent Department, 6600 Sears Tower, 233 South Wacker Drive, Chicago, IL, 60606, US

(21) Appl. No.: 10/769,334

(22) Filed: Jan. 30, 2004

(30) Foreign Application Priority Data
Jan. 30, 2003 (DE) 103 03 717.9

(51) Int. Cl. A61B 8/00

(52) U.S. Cl. 600/437; 128/903

(57) ABSTRACT

A patient monitor, particularly for emergency rooms, intensive care units, ORs or the like of clinics, containing hardware modules and software modules for processing signals from sensors that are attached to a patient, which are supplied via an interface, also has hardware modules and software modules for processing signals of an ultrasound probe that are supplied via an interface.

### BACKGROUND OF THE INVENTION

1. Field of the Invention

The invention relates to a patient monitor, particularly for emergency rooms, intensive care stations, ORs or the like in clinics, containing hardware and software modules for processing signals from sensors that are attached to a patient and that are supplied via an interface.

2. Description of the Prior Art

Patient monitors with which the patient can be continuously observed are used in clinics for emergency patients and in recovery rooms. The patient can be connected to the monitors by a series of sensors, which can also be connected to a recovery room.

Simple ultrasound devices also have been increasingly recognized as useful in patient diagnosis, for instance in order to monitor the needle guidance during puncture of the femoral and brachial arteries. These ultrasound devices also can be used for the simple monitoring of heart functions. It is still standard practice today to take a daily X-ray of a patient in intensive care in order to determine whether fluid has collected in the lungs. These X-rays could be replaced in part by ultrasound examinations, so that the patient is not exposed to radiation unnecessarily.

Unfortunately, such ultrasound imaging systems are only rarely available in the clinic rooms mentioned above, due to the additional space that is required and the high price. Portable ultrasound devices are known. JP 2002272740 A (abstract), for example, describes an ultrasound device combined with an electrocardiograph, a printer, and a monitor, i.e. an ultrasound device that is complex and difficult to handle. Known devices of this type are so unwieldy that they cannot be carried without difficulty and they do not leave space for the monitor next at the multitude of devices of an intensive care unit bed. On the other hand, the monitors in smaller ultrasound devices are smaller than the palm of a hand, so that details are no longer reasonably discernible, which makes these miniature devices unsuitable for use in intensive care units.

### SUMMARY OF THE INVENTION

An object of the invention is to provide a device that allows relatively simple and inexpensive ultrasound examinations to be performed in connection with intensive care patients that are being monitored by patient monitors, without having to accept the offsetting disadvantages of the above described known systems in exchange.

This object is achieved in accordance with the invention by a patient monitor of the above described type having hardware and software modules installed in the patient monitor for processing signals of an ultrasound probe that are supplied via an interface, the patient monitor being constructed modularly to facilitate this installation.

With a relatively minor upgrading of an existing patient monitor according to the invention, it becomes possible to make ultrasound examinations by means of a simple portable ultrasound probe with a connection to the patient monitor, with the hardware and software components that are needed for processing the ultrasound signals representing very simple and inexpensive components in relation to the overall construction of such a monitor. No extra screen is required, and above all, no space for setting up such a screen for an ultrasound device at the bed in the intensive care unit. The overall power supply and control of the monitor can be utilized for the ultrasound signal processing as well; therefore, it is just barely more expensive to furnish a number of patient monitors for all intensive care accommodations or recovery rooms of a hospital than to acquire one or two high-quality ultrasound devices with corresponding high-performance monitors. The inventive solution also has the advantage that it requires no additional space at the patient bed, because the physician can carry the ultrasound probe in his or her coat pocket like a stethoscope.

The ultrasound probe can be connected to the patient monitor via a socket of the monitor for attaching a connecting cord to the ultrasound probe or wirelessly via infrared or a blue tooth radio pathway.

### DESCRIPTION OF THE DRAWINGS

FIG. 1 is a schematic view of three patient monitors in an intensive care unit with a connection capability for an ultrasound probe that is carried by the physician, in accordance with the invention.

FIG. 2 is a system overview of the patient monitor with integrated ultrasound modules in accordance with the invention.

### DESCRIPTION OF THE PREFERRED EMBODIMENTS

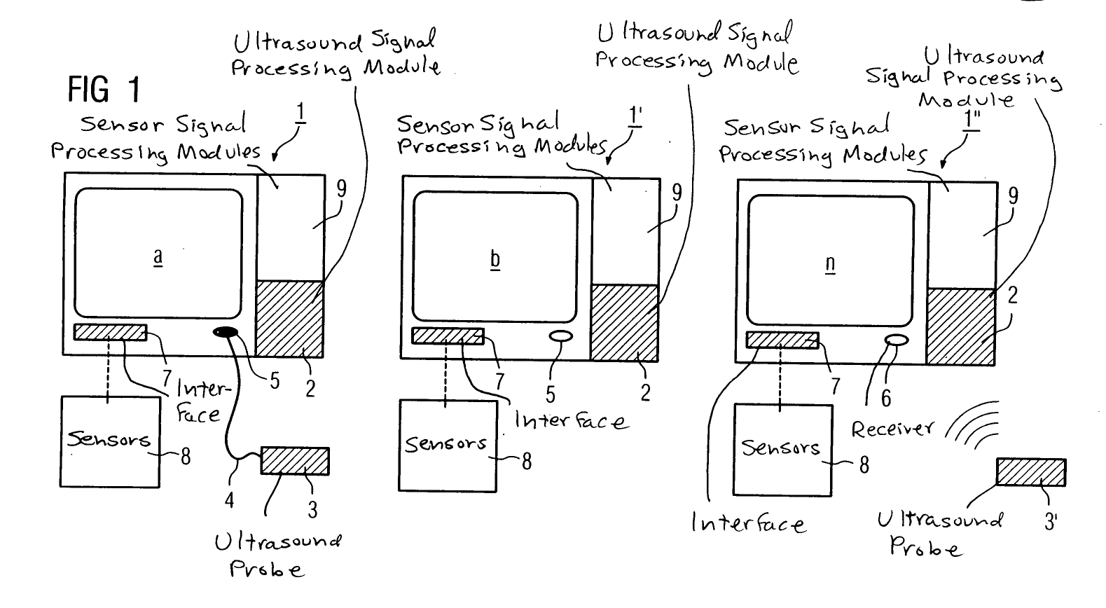

FIG. 1 schematically shows how separate patient monitors 1, 1′, 1″ are provided for each intensive care bed, three being represented in the exemplifying embodiment.

According to the invention, menus in the form of corresponding modules 2 are built into each patient monitor 1 in order to be able to process ultrasound signals from an ultrasound probe 3, which is carried by the physician as easily as a stethoscope, and display them on the patient monitor 1. To that end, in the exemplary embodiment according to FIG. 1, a connecting cable 4 with a plug portion is provided, which is insertable into a socket 5 of the patient monitor 1 in order to effectuate the connection to the respective monitor 1. As represented in the example of patient monitor 1″, a wireless connection by means of infrared or by means of a blue tooth radio path could also be provided instead of this connection via cable 4. The ultrasound probe 3′ transmits the data by means of a built-in transmitter to a receiver 6 that is integrated in the patient monitor 1″.

Various sensors 8 that are attached to the patients are connected to the respective patient monitors 1 to 1″ via a sensor/data interface 7 by means of the connections represented as dotted lines, in order to effectuate the desired optimally uninterrupted monitoring of the status of the patient with the aid of such monitors 1 to 1″. In order to be able to perform this task, which is an ordinary task for patient monitors 1 to 1″, they contain menus in the form of corresponding modules 9 for controlling the patient monitors 1 to 1″.

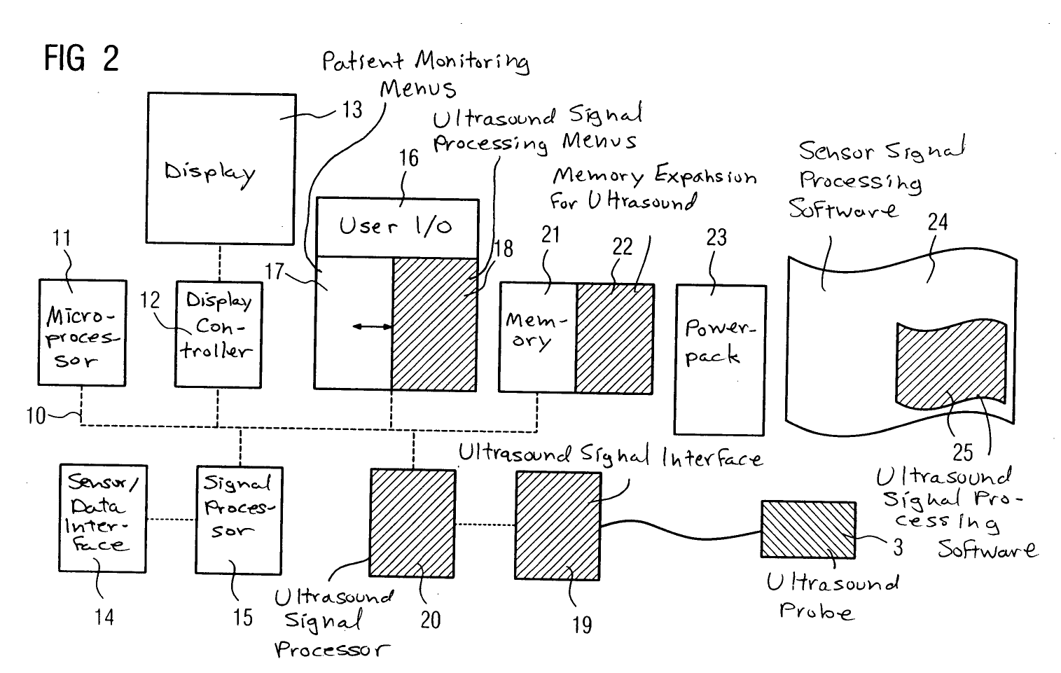

FIG. 2 represents a system overview of such a patent monitor 1 to 1″ with integrated ultrasound monitors. In a schematic block circuit diagram of a patient monitor 1 to 1″, the components and modules indicated by hatching are those components and modules which must be retrofitted in addition to the assemblies that are normally provided in order to effectuate the inventive enhancement of the patient monitor function for ultrasound examinations.

The patient monitors 1 to 1″ are provided with a bus 10 to which a microprocessor 11 is connected for controlling all the devices that are connected to the bus 10 and their routines. Connected to the bus 10 via a display controller 12 is a display 13 for the patient monitor functions, on which curves, data, and ultrasound images can be displayed, for example. The data of the sensors 8 that are attached to the patient are supplied, via a sensor/data interface 14 for the patient monitoring, to a signal processing device 15 that is connected to the bus 10. Menus 17 for patient monitoring and menus 18 for an ultrasound signal processing device 20 are retrievable by means of a user input/output 16, from which commands are sent to the corresponding device via the bus 10. The ultrasound probe 3 is connected, via an ultrasound-sensor/data interface 19, to the ultrasound signal processing unit 20 that is linked to the bus 10. Also linked to the bus 10 is a memory 21 with a memory expansion 22 for the ultrasound components. The patient monitor 1 is further provided with a power pack 23 for the system components. The sequence of the processes in the patient monitor 1 is controlled by the system software 24 for the patient monitor, which has been expanded by a system software 25 for the ultrasound signal processing unit 20.

Although modifications and changes may be suggested by those skilled in the art, it is the intention of the inventor to embody within the patent warranted hereon all changes and modifications as reasonably and properly come within the scope of his contribution to the art.

I claim as my invention:

1. A patient monitor comprising:
a plurality of first hardware modules and first software modules connected to sensors adapted for attachment to a patient, for processing signals from said sensors;
an interface to which said sensors are electrically connected;
an ultrasound probe; and
a second hardware module and a second software module and a further interface, said further interface communicating with said ultrasound probe to receive signals from said ultrasound probe, and said second hardware module and said second software module processing said signals from said ultrasound probe.

2. A patient monitor as claimed in claim 1 wherein said interface and said further interface allow addition of further hardware and software modules.

3. A patient monitor as claimed in claim 1 comprising a cable connecting said ultrasound probe to a socket at said further interface.

4. A patient monitor as claimed in claim 1 wherein said further interface is an interface selected from the group consisting of an infrared interface and a blue tooth radio path interface for wirelessly communicating with said ultrasound probe.
